(19) United States
(12) Patent Application Publication
(10) Pub. No.: US 2026/0272586 A1
(43) Pub. Date: Sep. 17, 2026

(54) SYSTEMS AND METHODS FOR MONITORING VIBRATION IN SURGICAL ROBOTS

(71) Applicant: Auris Health, Inc., Santa Clara, CA (US)

(72) Inventors: David MONTEVERDE, Redwood City, CA (US); Victor TORRES-BORRERO, Redwood City, CA (US); Nicholas PATRONIK, Redwood City, CA (US); Nicholas CHEUNG, Redwood City, CA (US)

(21) Appl. No.: 19/666,885

(22) Filed: May 4, 2026

Related U.S. Application Data
(63) Continuation of application No. PCT/IB2024/060978, filed on Nov. 6, 2024.
(60) Provisional application No. 63/596,553, filed on Nov. 6, 2023.

(51) Int. Cl. A61B 34/37; A61B 34/00; A61B 34/10; A61B 34/20; B25J 9/16; G16H 40/40

(52) U.S. Cl. CPC A61B 34/37; A61B 34/74; B25J 9/1653; B25J 9/1674; G16H 40/40; A61B 2034/102; A61B 2034/107; A61B 2034/2055; A61B 2034/2072

(57) ABSTRACT

A system for monitoring vibration induced in a surgical robot includes an end-effector having a first marker coupled thereto, a human interface device (HID) for actuating the end-effector, and a processor communicatively coupled to the HID. The processor is configured to actuate the end-effector based on an input provided by the HID, and detect an actual position of the first marker attached to the end-effector. In addition, the processor is configured to determine an expected position of the marker based on a kinematic model of the surgical robot and the input provided by the HID, and determine a position error between the actual position of the first marker and the expected position of the first marker.

### PRIORITY

This application is a continuation of International Application No. PCT/IB2024/060978, filed Nov. 6, 2024, entitled “SYSTEMS AND METHODS FOR MONITORING VIBRATION IN SURGICAL ROBOTS,” which claims priority to U.S. Provisional Application No. 63/596,553, filed Nov. 6, 2023, entitled “SYSTEMS AND METHODS FOR MONITORING VIBRATION IN SURGICAL ROBOTS,” the disclosures of which are incorporated by reference herein, in their entireties.

### TECHNICAL FIELD

The present disclosure pertains to the field of surgical robots, particularly those employed for minimally invasive and non-invasive surgical procedures.

### BACKGROUND

Surgical robots have become increasingly prevalent in modern medicine, offering improved surgical precision, reduced invasiveness, and enhanced dexterity for surgeons. These systems commonly employ one or more robotic arms equipped with various surgical instruments to perform intricate tasks within a patient's body. Such tasks may include, among other things, tissue manipulation, suturing, cauterization, and precise incisions.

### SUMMARY

An embodiment of a method comprises (a) coupling a first marker to an end-effector of a surgical robot, (b) actuating the end-effector based on an input provided by a human interface device (HID) of the surgical robot, (c) detecting an actual position of the first marker coupled to the end-effector, (d) determining an expected position of the first marker based on a kinematic model of the surgical robot and the input provided by the HID, and (e) determining a position error between the actual position of the first marker and the expected position of the first marker. In some embodiments, the method comprises terminating the actuation of the end-effector in response to the position error exceeding a predefined threshold. In some embodiments, the method comprises coupling a second marker to the HID. In certain embodiments, the input from the HID is generated in response to moving the HID along a predefined trajectory. In certain embodiments, the first marker comprises an optical marker, and (c) comprises optically monitoring by an optical device the position of the optical marker as the end-effector is actuated. In some embodiments, the end-effector moves along a predefined test trajectory based on the input provided by the HID. In some embodiments, the expected position corresponds to a position along a predefined intended trajectory of the first marker. In certain embodiments, the method comprises determining at least a 90th percentile of the position error across an actual trajectory of the first marker comprising the actual position. In certain embodiments, the method comprises defining an error metric corresponding to (E_mean+2xE_SD), where E_mean comprises a mean of the position error, x comprises a predefined constant, and E_SD comprises a standard deviation of the position error, and determining an acceptability of a vibration induced in the surgical robot by determining whether the error metric meets or exceeds a predefined error threshold. In some embodiments, the method comprises determining an acceptability of a vibration induced in the surgical robot by determining whether the position error meets or exceeds a predefined error threshold. In some embodiments, the method comprises inferring a position error of the end-effector based on the position error between the actual position of the first marker and the expected position of the first marker determined at (e). In certain embodiments, the actual position comprises a first actual position of the first marker corresponding to a first time segment occurring as the end-effector is actuated, and the expected position comprises a first expected position corresponding to the first time segment and based on the kinematic model, and the input provided by the HID during the first time segment. In certain embodiments, the method comprises determining a second expected position of the first marker corresponding to a second time segment and based on the kinematic model and and the input provided by the HID during the second time segment, wherein the second time segment follows the first time segment, and determining a second position error between a second actual position of the first marker corresponding to the second time segment and the second expected position of the first marker.

An embodiment of a system for monitoring vibration induced in a surgical robot comprises an end-effector having a first marker coupled thereto, a human interface device (HID) for actuating the end-effector, and a processor communicatively coupled to the HID, wherein the processor is configured to actuate the end-effector based on an input provided by the HID, detect an actual position of the first marker attached to the end-effector, determine an expected position of the marker based on a kinematic model of the surgical robot and the input provided by the HID, and determine a position error between the actual position of the first marker and the expected position of the first marker. In some embodiments, the processor is configured to determine at least a 90th percentile of the position error across an actual trajectory of the first marker comprising the actual position. In some embodiments, the processor is configured to define an error metric corresponding to (E_mean+KE_SD), where E_mean comprises a mean of the position error, K comprises a predefined constant, and E_SD comprises a standard deviation of the position error, and determine an acceptability of a vibration induced in the surgical robot by determining whether the error metric meets or exceeds a predefined error threshold. In certain embodiments, the processor is configured to determine an acceptability of the vibration induced in either the surgical robot by determining whether the position error meets or exceeds a predefined error threshold. In certain embodiments, the first marker comprises an optical marker. In some embodiments, the system comprises a second marker coupled to the HID. In some embodiments, the first marker comprises an optical marker, and the processor is configured to monitor by an optical device communicatively coupled to the processor the position of the optical marker as the end-effector is actuated.

An embodiment of a method comprises (a) actuating a first end-effector of the surgical robot based on a first input provided by a first human interface device (HID) of the surgical robot, (b) coupling a first marker to a second end-effector of a surgical robot, (c) detecting an actual position of the first marker coupled to the second end-effector, (d) determining an expected position of the first marker, and (e) determining a position error between the actual position of the first marker and the expected position of the first marker. In certain embodiments, (a) comprises at least one of moving a surgical table to which the first end-effector is coupled and repositioning a robotic arm to which the first end-effector is coupled. In certain embodiments, the expected position comprises a single stationary position of the first marker. In some embodiments, the method comprises actuating the second end-effector based on a second input provided by a second HID of the surgical robot, wherein the expected position is based on a kinematic model of the surgical robot and the second input provided by the second HID.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates vibrations in an exemplary surgical robot according to some embodiments.

FIG. 2 illustrates an exemplary surgical robot according to some embodiments.

FIG. 3 illustrates components of the surgical robot of FIG. 2 in accordance with some embodiments.

FIG. 4 illustrates an exemplary controller of the surgical robot of FIG. 2 in accordance with some embodiments.

FIG. 5A illustrates a side view of an exemplary robotic arm of the surgical robot of FIG. 2, with a medical tool loaded and with the robotic arm in an articulated state.

FIG. 5B illustrates a side view of the robotic arm of FIG. 5A, without a medical tool loaded and with the robotic arm in a straightened state.

FIG. 5C illustrates a perspective view of the robotic arm of FIG. 5A, with a medical tool loaded and with the robotic arm in an articulated state.

FIG. 6 illustrates an exemplary system for monitoring vibration in the surgical robot of FIG. 2 according to some embodiments.

FIG. 7 illustrates a graph depicting actual positions and intended positions of a marker over time in the vibration monitoring system of FIG. 6, according to some embodiments.

FIG. 8 illustrates a graph depicting an actual trajectory of a marker and an intended test trajectory of the marker in the vibration monitoring system of FIG. 6, according to some embodiments.

FIG. 9 illustrates a graph depicting position error magnitude of a marker over time in the vibration monitoring system of FIG. 6, according to some embodiments.

FIG. 10 illustrates a graph depicting an actual trajectory of a marker in the vibration monitoring system of FIG. 6, according to some embodiments.

FIG. 11 illustrates a graph depicting position error magnitude of a marker over time in the vibration monitoring system of FIG. 6, according to some embodiments.

FIG. 12 illustrates a flowchart of an exemplary method for monitoring vibration in a surgical robot using the vibration monitoring system of FIG. 6, according to some embodiments.

FIG. 13 illustrates a flowchart of another exemplary method for monitoring vibration in a surgical robot using the vibration monitoring system of FIG. 6, according to some embodiments.

FIG. 14 is a schematic diagram illustrating electronic components of the surgical robot of FIG. 2, in accordance with some embodiments.

### DETAILED DESCRIPTION

This application discloses systems and methods for monitoring vibration in surgical robots used in the performance of medical procedures such as minimally invasive and non-invasive surgical operations. As will be discussed further, vibration induced in a surgical robot during operation may interfere with a user's ability to perform a surgical operation using the surgical robot. Such vibration can arise from various sources within the system or due to external inputs to the system. For example, vibration can arise from movement of the robotic arms of the surgical robot, and physical contact with external objects such as the patient's body, operators of the surgical robot, etc. Vibration induced in the surgical robot may manifest as static position errors, oscillations, or tremors in the robotic arms, affecting their stability, accuracy, and overall performance.

Vibrations in surgical robots can, if not properly addressed during the design and testing of the surgical robot, compromise the accuracy and precision of surgical procedures conducted using the surgical robot. For instance, the slightest deviation of a surgical instrument manipulated by the surgical robot (e.g., via a robotic arm of the system) from an intended position or path can have serious consequences for patient safety and the success of the respective surgical procedure. Therefore, addressing vibration-related issues is critical to ensuring the surgical robot's ability to perform precise surgical tasks.

Here we describe a few specific examples of the issues which may obtain from excessive vibration in a surgical robot. Excessive vibration can lead to unintended tissue trauma and damage during surgical procedures. Excessive vibration may also result in excessive position errors of the surgical instrument carried by a given robotic arm or oscillations which in return may cause unintended tissue displacement or injury. In addition, prolonged exposure to excessive vibration can cause discomfort and fatigue for the surgeon operating the surgical robot due to the difficulties encountered by the surgeon in properly positioning medical instruments manipulated by the surgical robot. Particularly, the need to compensate for vibration and maintain control over the robotic arms can lead to physical strain and reduced surgeon performance. Some other challenges of the excessive vibration is the disturbance of the precise control of surgical instruments in the operation room and while the surgeon is manipulating tissues, performing delicate maneuvers. These challenges can propagate through the system's mechanical components, potentially causing malfunctions, calibration errors, or other disruptions between system components.

In view of these issues, there is a need for innovative solutions that minimizes vibration induced in surgical robots during their operation, particularly given that scant attention has been given to addressing these issues in conventional surgical robots. Successfully addressing these challenges will enhance the safety, reliability, and performance of surgical robots. The present disclosure seeks to address these issues and provide a more effective way for the performance of minimally invasive and non-invasive surgical procedures.

Particularly, surgical robots are subject to various forms of vibration during operation conducted using the surgical robot. It may be understood that vibrations encountered by surgical robots during operation may be disruptive to a user of the surgical robot performing the operation. For example, vibrations encountered by surgical robots during operation may require significant mental compensation from the user in an attempt to correct against the encountered vibrations so the user may successfully execute the intended operation. Additionally, significant vibration encountered by a surgical robot during operation may render the continued performance of the operation unsafe (e.g., the vibrations may cause the user to make a mistake during the performance of the operation) or force the user to proceed at a slower pace, thereby undesirably prolonging the operation.

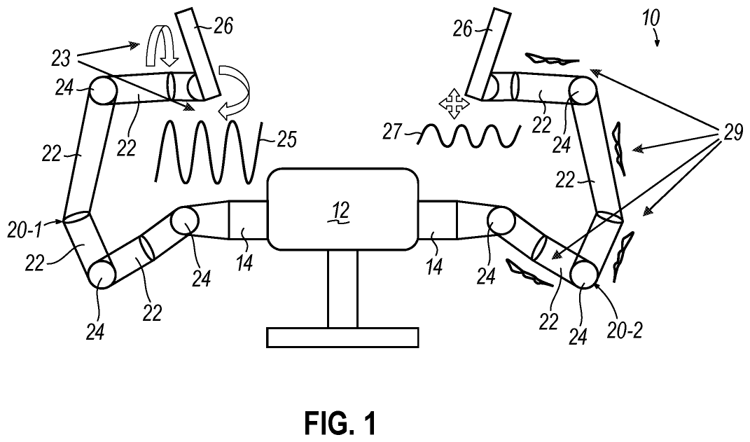

One form of vibration encountered by surgical robots during operation includes self-excitation. For example, and referring now to FIG. 1, an embodiment of a surgical robot 10 is shown, the surgical robot 10 is engaged in an operation and subject to vibration, including self-excitation. Particularly, in this exemplary embodiment, surgical robot 10 generally includes a base or a support structure 12 and a plurality of robotic arms 20 (indicated as 20-1 and 20-2 in FIG. 1 for clarity) coupled to the base 12. The base 12 may further include a pair of adapters or a plurality of connectors 14 coupled to a proximal ends of the pair of the robotic arms 20. Additionally, each robotic arm 20 includes a plurality of links 22 and a plurality of joints 24 coupled between the plurality of links 22 to provide robotic arms 20 with the desired number of degrees-of-freedom (DoFs). Further, each robotic arm 20 is coupled to an end-effector 26 such as a medical device or tool that is used by the surgical robot 10 to perform a medical or surgical operation.

As shown in FIG. 1, during the operation, one or more of the joints 24 of a first robotic arm 20-1 of the surgical robot 10 may be activated to intentionally move one or more of the corresponding links 22 of the first robotic arm 20-1 (indicated by arrows 23 in FIG. 1) resulting in unintended vibration or motion (indicated in FIG. 1 by numeral 25) in the activated robotic arm 20-1. It is necessary to note that the vibration or motion may sometimes be referred to as self-excitation of the activated robotic arm 20-1. In other words, self-excitation refers to vibration induced in a robotic arm of a surgical robot (e.g., one of the robotic arms 20 of surgical robot 10) by motion of the respective robotic arm in an activated state. As used herein, the term “activated state” with reference to a robotic arm is defined as meaning a state of the respective robotic arm in which at least one of the links comprising the robotic arm is in motion with respect to a proximal end of the robotic arm coupled to a base or other support structure.

Self-excitation of a respective robotic arm of a surgical robot may manifest as tracking error which can, in at least some instances, be particularly disruptive to a user of the surgical robot. Specifically, the term “tracking error” is defined herein as meaning the unintended motion of a robotic arm (or an end-effector coupled to the robotic arm) of a surgical robot when the robotic arm is in an activated state. In other words, tracking error corresponds to motion of a robotic arm in the activated state that is not intended by a user of the robotic arm. Tracking error may manifest as transient unintended motion at relatively high frequencies as compared with low frequency or quasi-static errors where high frequency unintended motion can be particularly difficult to account for by the user given the limited reaction time of human operators (e.g., the frequency of the tracking error may outpace the user's reaction time).

Beyond self-excitation as described above, another source of vibration that may result from the performance of an operation using a surgical robot is cross-talk. Particularly, the term “crosstalk” is defined herein as meaning when motion of a first robotic arm of a surgical robot in an activated state results in unintended motion of a second robotic arm (which may be stationary or in an activated state) of the surgical robot For example, the motion of the first robotic arm 20-1 of the surgical robot 10 shown in FIG. 1 results in the unintended motion of the end-effector 26 of the second robotic arm 20-2 (indicated in FIG. 1 by numeral 27). Particularly, forces and/or torques resulting from the intended motion 23 of the first robotic arm 20-1 my be transferred through the first robotic arm 20-1, the base 12, and the second robotic arm 20-2 (indicated by arrows 29 in FIG. 1), and to the end-effector 26 coupled to the second robotic arm 20-2 to produce the unintended motion or crosstalk 27. In addition to crosstalk, vibration may be transmitted from an underlying table or support structure to a robotic arm supported by the table. Such vibration, including crosstalk, may be particularly disruptive to a user of a surgical robot when the crosstalk manifests in a robotic arm carrying a sensor (e.g., a camera, an endoscope monitored by the user via the physician console 240) relied on by the user in controlling other robotic arms of the surgical robot that are currently being activated (e.g., in an activated state) by the user. Crosstalk manifested in the sensor may thus make it substantially more difficult for the user to accurately control the robotic arms in the activated state.

Vibration induced in a surgical robot, including self-excitation and cross-talk as described above, may cause a position error where an actual position of an end-effector manipulated by the surgical robot deviates from an intended position (e.g., as intended by a user of the surgical robot) of the end-effector. Deviation from the intended position by the end-effector may limit the precision of the end-effector in performing a surgical talk, risk collision between the end-effector and an external object, and generally reduce the usability of the surgical robot as described in greater detail above.

The exemplary systems and methods for monitoring vibrations in surgical robots described herein address the issues outlined above by determining a position error corresponding to a difference between an actual position of an end-effector of a surgical robot and an intended position of the end-effector. In some embodiments, the end-effector is actuated based on an input provided by a human interface device (HID) of the surgical robot. In some embodiments, the HID may comprise a haptic interface device. In certain embodiments, the intended or expected position of the end-effector may be determined based on a kinematic model of the surgical robot and the input provided by the HID. In some embodiments, the actual position of the end-effector may be monitored by a sensor such as an image sensor or camera. For example, an identifier or marker (e.g., an optical marker) may be coupled to the end-effector to facilitate the monitoring of the end-effector using the sensor.

In addition to determining the position error between the intended/expected position and the actual position of the end-effector, in some embodiments, the position error is compared with a predefined reference threshold or standard to assess the surgical robot's suitability for operation. For instance, surgical robots having a position error greater than the reference standard may be deemed unsuitable for operation, and the respective surgical robot may be repaired, refurbished, reconfigured, recalibrated, etc., prior to being retested to determine if the surgical robot is operating within desirable measurements.

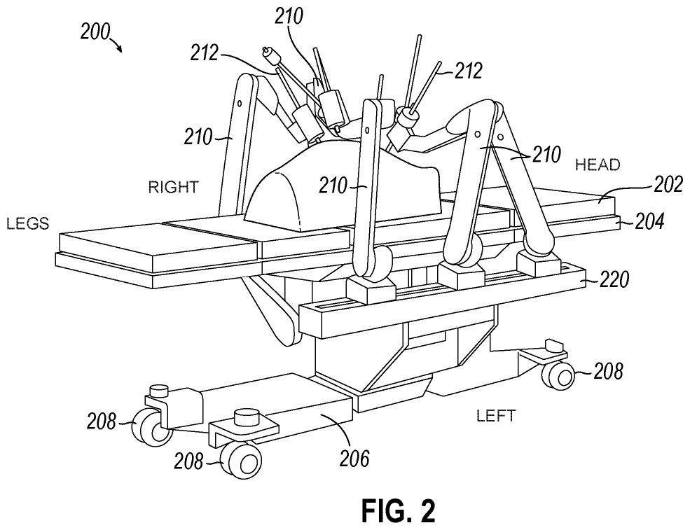

Referring now to FIG. 2, an exemplary robotic surgical or medical system 200 is illustrated. The surgical robot 200 comprises a patient platform 202 (e.g., a patient platform, a table, a bed) including a support 204 (e.g., a rigid frame). The two ends along the longitudinal length of the patient platform 202 are respectively referred to herein as “head” and “legs”. Additionally, the lateral two sides of the patient platform 202 are respectively referred to herein as “left” and “right.”

In addition to the patient platform 202, in this exemplary embodiment, the surgical robot 200 may also include a base 206 for supporting the surgical robot 200. The base 206 includes a plurality of wheels 208 that allow the surgical robot 200 to be easily movable or repositionable in a physical environment. In some embodiments, the wheels 208 are retractable, may be replaced with feet, or may be entirely omitted from the surgical robot 200 with the base 206 resting directly on the ground.

The surgical robot 200 includes one or more robotic arms 210. In some embodiments, the robotic arms 210 can be configured to perform robotic medical procedures including, for example, minimally invasive procedures such as laparoscopy. Additionally, although FIG. 2 illustrates five robotic arms 210, it should be appreciated that the surgical robot 200 may include any number of robotic arms 210, including less than five (e.g., four arms) or six or more.

The surgical robot 200 also includes one or more support rails 220 (e.g., adjustable arm support or an adjustable bar) that support the robotic arms 210. Each of the robotic arms 210 is supported on, and movably coupled to, a support rail 220, by a respective base joint of the robotic arm 210. In some embodiments, support rail 220 can provide several DoFs, including lift, lateral translation, tilt, etc. Each of the robotic arms 210 and/or the support rails 220 may also be referred to as a respective kinematic chain. Additionally, in this exemplary embodiment and as illustrated in FIG. 2, three robotic arms 210 are supported by the support rail 220 that is in the field of view of the figure which is located along the left side of the patient support platform. The two remaining robotic arms 210 are supported by another support rail 220 located along an opposing lateral side of the patient platform 202.

In some embodiments, support rails 220 provide a base position for one or more of the robotic arms 210 for a robotic medical procedure. A robotic arm 210 can be positioned relative to the patient platform 202 by translating the robotic arm 210 along a length of its underlying support rail 220 and/or by adjusting a position and/or orientation of the robotic arm 210 via one or more joints and/or links. In some embodiments, the pose of a given support rail 220 can be changed via manual manipulation, operation, and/or power assisted motion. For example, in some embodiments, the support rail 220 can be translated along a length of the patient platform 202. In certain embodiments, translation of the support rail 220 along a length of the patient platform 202 causes one or more of the robotic arms 210 supported by the support rail 220 to be simultaneously translated with the respective support rail 220 or relative to the support rail 220. In certain embodiments, the support rail 220 can be translated while keeping one or more of the robotic arms stationary with respect to the base 206 of the surgical robot 200. Additionally, in this exemplary embodiment, the support rail 220 is located along a length of the patient platform 202. In some embodiments, the support rail 220 may extend across a partial or full length of the patient platform 202, and/or across a partial or full width of the patient platform 202.

During a robotic medical procedure, one or more of the robotic arms 210 can also be configured to hold end-effectors (e.g., robotically controlled medical instruments or tools, such as an endoscope and/or any other instruments such as sensors, illumination instrument, cutting instrument, etc. that may be used during surgery), and/or be coupled to one or more accessories, including one or more cannulas, in accordance with some embodiments.

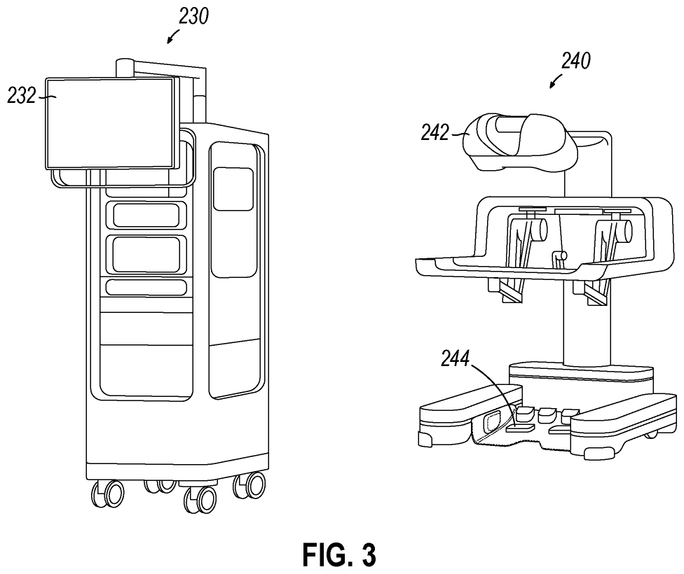

Referring to FIG. 3, in some embodiments, the surgical robots described herein (e.g., surgical robot 200 include a tower 230 (e.g., tower viewer) and/or a physician console 240 (or both). The tower 230 may provide support for controls, electronics, fluidics, optics, sensors, and/or power for the patient platform 202 and the physician console 240. In some embodiments, the tower 230 includes a display device 232 including a user interface for displaying a surgical view obtained by one or more cameras of the surgical robot and/or one or more notifications to an operator of the surgical robot. In some embodiments, the physician console 240 includes a display device 242 having a user interface usable by a physician operator for operating the patient platform 202. For example, the display device 242 may include a user interface for displaying surgical views obtained by one or more cameras of the surgical robot and/or one or more notifications to an operator of the surgical robot. The physician console 240 can provide both robotic controls and pre-operative and real-time information of a medical procedure to a physician operator. In some embodiments, the physician console 240 includes one or more input devices (e.g., buttons, switches, touch-sensitive surfaces, gimbals, etc.), such as, for example, a foot pedal 244. Additionally, in some embodiments, the physician console 240 includes one or more human interface devices (HIDs) that provide force and tactile feedback to a user as the user interacts with the physician console 240.

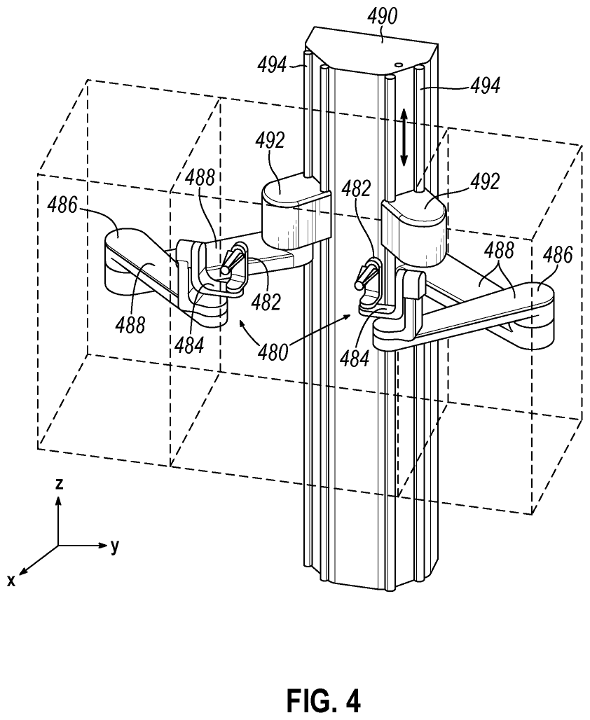

Referring to FIG. 4, in some embodiments, the surgical robot described herein includes a HID 480 for actuating the end-effectors of the surgical robot. In this exemplary embodiment, HID 480 comprises a hybrid HID that can have both impedance and admittance control. In other embodiments, HID 480 can utilize just impedance or passive control. In still other embodiments, HID 480 can utilize just admittance control. By being a hybrid HID, the HID 480 advantageously can have a lower perceived inertia while in use.

As shown in FIG. 4, in this exemplary embodiment, the HID 480 is configured to allow manipulation of a pair of end-effectors, and includes a pair of handles 482. Handles 482 may be operated by a human operator (e.g., a physician) or by an additional robotic arm (e.g., via a grasper or handle of the additional robotic arm). Each of the handles 482 is connected to a gimbal 484 of the HID 480. In addition, each gimbal 484 is connected to a positioning platform 486 of HID 480. In this exemplary embodiment, each positioning platform 486 comprises a robotic arm such as a SCARA arm (selective compliance assembly robot arm) 488 coupled to a column 490 of HID 480 by a prismatic joint 492 of HID 480. The prismatic joints 492 are configured to translate along the column 490 (e.g., along rails 494 of column 490) to allow each of the handles 482 to be translated in the z-direction shown in FIG. 4, providing a first degree of freedom. In addition, the robotic arm 488 is configured to allow motion of the handle 482 in an x-y plane shown in FIG. 4 providing two additional degrees of freedom.

In some embodiments, one or more load cells are positioned in or on HID 480. For example, in some embodiments, a load cell is positioned in the body of each of the gimbals 484. By providing a load cell, portions of HID 480 are capable of operating under admittance control, thereby advantageously reducing the perceived inertia of HID 480 while in use. In some embodiments, the positioning platform 486 is configured for admittance control, while the gimbal 484 is configured for impedance control. In other embodiments, the gimbal 484 is configured for admittance control, while the positioning platform 486 is configured for impedance control. Accordingly, for some embodiments, the translational or positional degrees of freedom of the positioning platform 486 can rely on admittance control, while the rotational degrees of freedom of the gimbal 484 rely on impedance control.

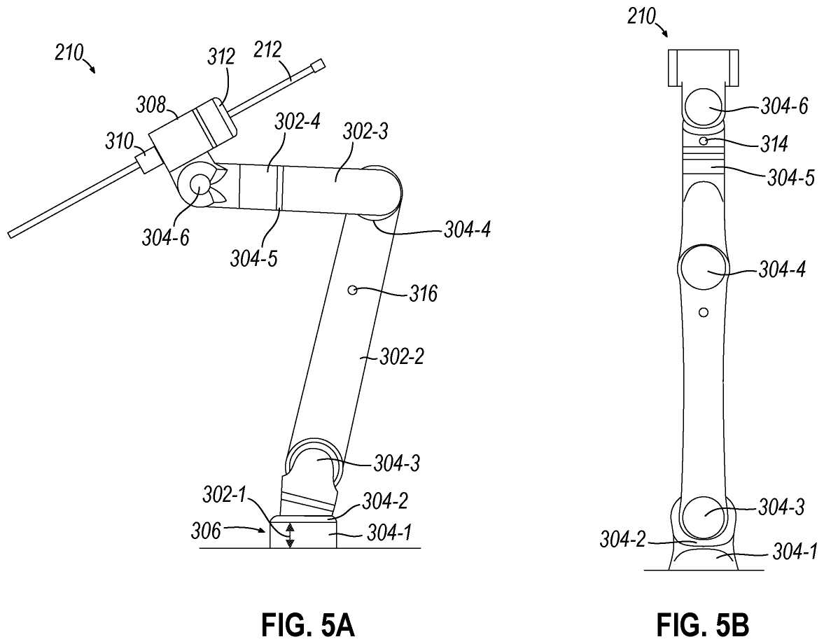

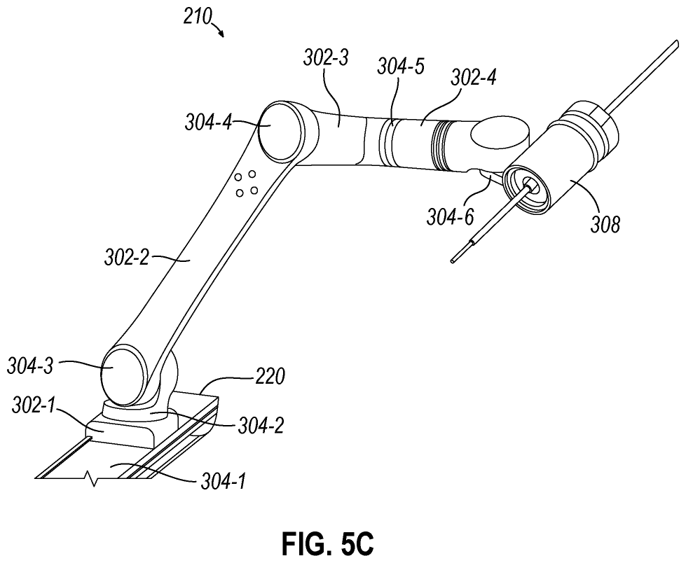

Referring now to FIGS. 5A-5C, different views of an exemplary robotic arm 210 are shown according to some embodiments. Particularly, in this exemplary embodiment, robotic arm 210 includes a plurality of links 302 (e.g., linkages), indicated as 302-1 through 302-4 in FIGS. 5A-5C, connected by one or more joints 304 (indicated as 304-1 through 304-6 in FIGS. 5A-5C). It may be understood that each of the joints 304 includes one or more DoFs.

In FIG. 5A, the joints 304 include a first joint 304-1 (e.g., a base joint or an A0 joint) located at or near a base 306 of the robotic arm 210. In some embodiments, the base joint 304-1 comprises a prismatic joint that allows the robotic arm 210 to translate along the support rail 220. The joints 304 also include a second joint 304-2 which, in some embodiments, rotates with respect to the base joint 304-1. The joints 304 also include a third joint 304-3 that is connected to a first end of link 302-2 and which, in some embodiments, includes multiple DoFs and facilitates both tilt and rotation of the link 302-2 tilt with respect to the third joint 304-3. In addition, joints 304 include a fourth joint 304-4 that is connected to a second end of the link 302-2. In some embodiments, the joint 304-4 comprises an elbow joint that connects the link 302-2 to the link 302-3. In this exemplary embodiment, the joints 304 further include a pair of joints 304-5 (e.g., a wrist roll joint) and 304-6 (e.g., a wrist pitch joint), which is located on a distal portion of the robotic arm 210.

In this exemplary embodiment, a proximal end of the robotic arm 210 is connected to a base 306 and a distal end of the robotic arm 210 is connected to device manipulator or driver such as, for example, an advanced device manipulator (ADM) 308 (e.g., a tool driver, an instrument driver). The ADM 308 may be configured to control the positioning and manipulation of a medical instrument s (e.g., a tool, a scope, etc.). For example, in some embodiments, the links 302 may be detachably coupled to a medical tool 212 (e.g., to facilitate ease of mounting and dismounting of the medical tool 212 from the robotic arm 210) carried by the ADM 308. The joints 304 provide the robotic arm 210 with a plurality of DoFs required to facilitate control of the medical tool 212 via the ADM 308.

The robotic arm 210 may also include a cannula sensor 310 for detecting the presence or proximity of a cannula to the robotic arm 210. In some embodiments, the robotic arm 210 is placed in a docked state (e.g., docked position) when the cannula sensor 310 detects the presence of a cannula (e.g., via one or more processors of the surgical robot 200). Conversely, when no cannula is detected by the cannula sensor 310, the robotic arm 210 is placed in an undocked state (e.g., undocked position).

In some embodiments, and as shown particularly in FIG. 5A, the robotic arm 210 includes a control input or button 312 (e.g., an annular button, or other types of controls, etc.) that can be used to place the robotic arm 210 in an admittance mode (e.g., by depressing the button 312). The admittance mode is also referred to as an admittance scheme or admittance control. In the admittance mode, the surgical robot 200 measures forces and/or torques (e.g., imparted on the robotic arm 210) and outputs corresponding velocities and/or positions. In some embodiments, the robotic arm 210 can be manually manipulated by a user (e.g., during a set-up procedure, or in between procedures, etc.) in the admittance mode. In some instances, by using the admittance mode, a user of robotic arm 210 need not overcome all of the inertia in the surgical robot 200 to move the robotic arm 210. For example, in the admittance mode, the surgical robot 200 can measure the force and assist the operator in moving the robotic arm 210 by driving one or more motors associated with the robotic arm 210 when the operator imparts a force on the arm, thereby producing the desired velocities and/or positions of the robotic arm 210.

In some embodiments, the robotic arm 210 includes a second input or button 314 (e.g., a push button) that is distinct from the button 312 shown in FIG. 5A (referred to now as the first button 312), for placing the robotic arm 210 in an impedance mode (e.g., by a single press or continuous press and hold of the second button 314). The impedance mode is also referred to as impedance scheme or impedance control. In the impedance mode, the surgical robot 200 measures displacements (e.g., changes in position and velocity) and outputs forces and/or torques to facilitate manual movement of the robotic arm 210. In some embodiments, the robotic arm 210 can be manually manipulated by a user (e.g., during a set-up procedure) in the impedance mode. In some embodiments, under the impedance mode, the operator's movement of one part of a robotic arm 210 may cause motion in one or more joints and/or links throughout the robotic arm 210.

In some embodiments, for admittance control, a force sensor or load cell measures the force that the operator is applying to the robotic arm 210 and moves the robotic arm 210 in a way that feels light. Admittance control may feel lighter than impedance control because, under admittance control, one can hide the perceived inertia of the robotic arm 210 through the motorized actuation of the joints 304 thereof. Conversely, in some embodiments, the user is responsible for most if not all mass acceleration using impedance control.

In some circumstances, depending on the position of the robotic arm 210 relative to the user, it may be inconvenient to reach the first button 312 and/or the second button 314 to activate a manual manipulating mode (e.g., the admittance mode and/or the impedance mode). Accordingly, under these circumstances, it may be convenient for the operator to trigger the manual manipulation mode other than by buttons. Additionally, in some embodiments, the robotic arm 210 includes a single button (e.g., the button 312 or 314) that can be used to place the robotic arm 210 in the admittance mode and/or the impedance mode (e.g., by using different presses, such as a long press, a short press, press and hold etc.). In some embodiments, the robotic arm 210 can be placed in impedance mode by a user pushing on arm linkages (e.g., the links 302) and/or joints (e.g., the joints 304) and overcoming a force threshold. In some embodiments, the admittance mode and the impedance mode are common in that they both allow the user to grab the robotic arm 210 and command motion thereof by directly and physically interfacing with the robotic arm 210.

In some embodiments, the robotic arm 210 includes an input control for activating an arm follow mode. For example, in some embodiments, the robotic arm 210 includes a designated touch point located on a link 302 or a joint 304 of the robotic arm 210 (e.g., an outer shell of the link 302 or a button 316). User interaction (e.g., user touch) with the designated touch point activates the arm follow mode. In some embodiments, the robotic arm 210 includes multiple touch points in which user interaction with any (e.g., one or more) of the touch points activates the arm follow mode of the robotic arm 210

During a medical procedure, it may be desired to have the ADM 308 of the robotic arm 210 and/or a remote center of motion (RCM) of the medical tool 212 coupled thereto kept in a static pose (e.g., position and/or orientation). An RCM may refer to a point in space where a cannula or other access port through which a medical tool 212 is inserted is motion constrained. In some embodiments, the medical tool 212 includes an end-effector that is inserted through an incision or natural orifice of a patient while maintaining the RCM. In some embodiments, the medical tool 212 includes an end-effector that is in a retracted state during a setup process of the surgical robot.

In some circumstances, the surgical robot 200 may be configured to move one or more links 302 of the robotic arm 210 within a “null space” to avoid collisions with nearby objects (e.g., other robotic arms), while the ADM 308 of the robotic arm 210 and/or the RCM are maintained in their respective poses. The null space may be viewed as the set of joint states through which a robotic arm 210 is permitted to move which does not result in movement of the ADM 308 and/or RCM, thereby maintaining the position and/or the orientation of the medical tool 212 (e.g., within a patient). In some embodiments, a robotic arm 210 may have multiple positions and/or configurations available for each pose of the ADM 308.

For a robotic arm 210 to move the medical tool 212 to a desired pose in space, in certain embodiments, the robotic arm 210 is provisioned with at least six DoFs-three DoFs for translation (e.g., X, Y, and Z positions) and three DoFs for rotation (e.g., yaw, pitch, and roll). In some embodiments, each joint 304 may provide the robotic arm 210 with a single DoF, and thus, the robotic arm 210 may have at least six joints to achieve freedom of motion to position the ADM 308 at any pose in space. To further maintain the ADM 308 or the RCM of the robotic arm 210 in a desired pose, the robotic arm 210 may further be provided with at least one additional “redundant joint.” Thus, in certain embodiments, the system may include a robotic arm 210 having at least seven joints 304, providing the robotic arm 210 with at least seven DoFs. In some embodiments, the robotic arm 210 may include a subset of joints 304 each having more than one DoF thereby achieving the additional DoFs for null space motion. Depending on the embodiments, the robotic arm 210 may have a greater or fewer number of DoFs.

Furthermore, in some embodiments, the support rail 220 may provide several additional DoFs, including lift, lateral translation, tilt, etc. Thus, depending on the embodiments, a surgical robot may have many more robotically controlled DoFs beyond just those in the robotic arms 210 to provide for null space movement and/or collision avoidance. In a respective embodiment of these embodiments, the end-effectors of one or more robotic arms (and any tools or instruments coupled thereto) and a remote center along the axis of the tool can advantageously maintain in pose and/or position within a patient. Additionally, it may be understood that a robotic arm 210 having at least one redundant DoF has at least one more DoF than the minimum number of DoFs for performing a given task. For example, a robotic arm 210 can have at least seven DoFs, where one of the joints 304 of the robotic arm 210 can be considered a redundant joint, in accordance with some embodiments. The one or more redundant joints can allow the robotic arm 210 to move in a null space to both maintain the pose of the ADM 308 and a position of an RCM and avoid collision(s) with other robotic arms or objects.

As described further above, surgical robots are subject to various forms of vibration during operation conducted using the surgical robot including self-excitation, cross-talk, etc. It may be understood that vibrations encountered by surgical robots during operation may be disruptive to a user of the surgical robot performing the operation. Particularly, vibration induced in the surgical robot may cause an actual position of an end-effector of the surgical robot to deviate undesirably from an intended or expected position of the end-effector, reducing the usability of the surgical robot. To address the issues of vibration induced in a surgical robot engaged in operation, embodiments of systems for monitoring vibration induced in a surgical robot are described herein. Particularly, the systems for monitoring vibration disclosed herein may determine a position error between an actual position of an end-effector via a marker (e.g., a marker coupled to the end-effector of the surgical robot) and an expected position of the end-effector. As used herein, the term “position error” refers to both errors in the position of the component and/or errors in the motion of the component. For example, in some embodiments, the position error of the component refers to a difference between an actual velocity and an intended velocity of the component, an actual acceleration and an intended acceleration of the component. It may be understood that the determination of the position error is performed, in at least some embodiments, by a processor (e.g., processors 380) in response to the processor executing instructions stored on non-transitory storage medium or memory connected to the processor. To state in other words, in at least some embodiments, the determination of the position error is performed by a computer or computing system comprising a processor and non-transitory storage medium or memory storing instructions executable by the processor.

The actual position of the marker may be detected as an end-effector (coupled to the marker) is actuated based on an input provided by a HID (e.g., HID 480 shown in FIG. 4) of the surgical robot (e.g., surgical robot 200). The input may be applied to the HID either manually by a human operator of the HID, via the HID itself based on encoded instructions provided to the HID, and/or via a robotic arm operating the HID. In addition, the expected position of the marker is determined based on a kinematic model of the surgical robot and the input provided by the HID. In some embodiments, the kinematic model may comprise a forward kinematic model of the surgical robot. The forward kinematic model may utilize kinematic equations of the surgical robot to determine the position of an end-effector from known parameters of the surgical robot. To state in other words, the forward kinematic model of the surgical robot may determine the pose of an end-effector (e.g., end-effector 213) of the surgical robot based on the kinematic chain of the surgical robot.

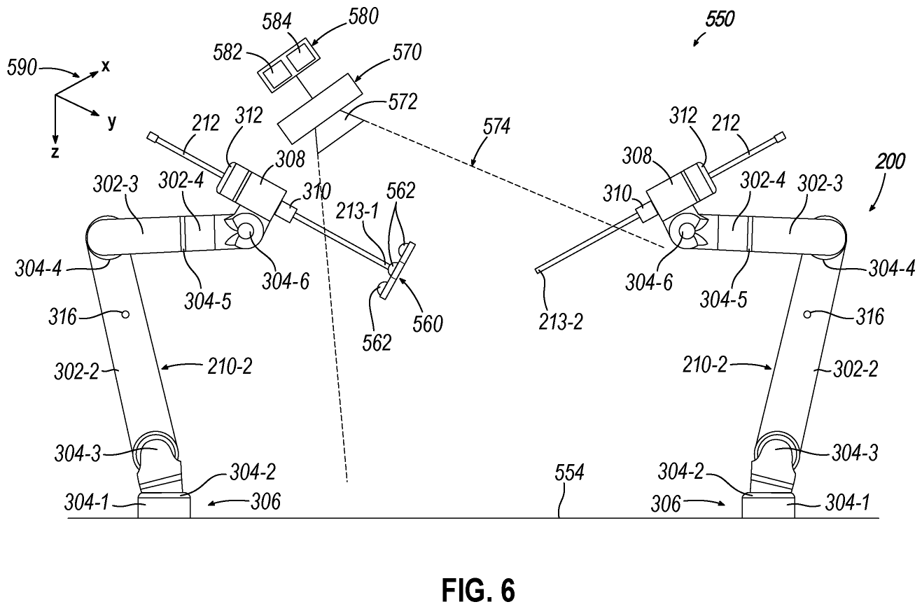

Referring now to FIG. 6, a vibration monitoring system 550 for monitoring vibration induced in surgical robot 200 is shown. In this exemplary embodiment, vibration monitoring system 550 includes a surgical robot 200 including at least a pair of robotic arms 210 (indicated as robotic arms 210-1 and 201-2 in FIG. 6) coupled or attached to a support structure 554. Although not shown in FIG. 6, it may be understood that at least some of the functionality of surgical robot 200 may be performed by one or more processors (e.g., processors 380) of a robotic control system. In some embodiments, the robotic control system is a surgical robot (e.g., surgical robot 200). In some embodiments, the robotic control system is an operation console (e.g., physician console 240) configured to control one or more robotic arms (e.g., robotic arm 210) of a surgical robot. The robotic control system includes memory (e.g., memory 382) storing instructions for execution by the one or more processors.

As described above, robotic arms 210 may be coupled to a medical tool 212 at a distal end thereof. A distal end or tip 213-1 of medical tool 212 defines an end-effector of the medical tool 212 and thus also may be referred to herein as end-effector 213. For example, the marker 560 may be manually attached to the end-effector 213 of medical tool 212 by a user of vibration monitoring system 550. In this exemplary embodiment, FIG. 6 particularly illustrates a first end-effector 213-1 coupled to robotic arm 210-1 and a second end-effector 213-2 similarly coupled to robotic arm 210-2 of surgical robot 200.

In this exemplary embodiment, the marker 560 includes one or more visual identifiers 562 configured to be easily and conveniently identified by an optical sensor or camera. For example, each visual identifier 562 may comprise a visually distinct color (e.g., a bright primary color), geometry, or other visually identifiable feature that is not otherwise present in the proximity of vibration monitoring system 550 to allow the position of each visual identifier 562 to be easily tracked by the optical sensor.

In this exemplary embodiment, the vibration monitoring system 550 of FIG. 6 additionally includes an optical sensor or camera 570 and a computer system 580 connected to the camera 570. The camera 570 includes a lens 572 which defines a field of view (FoV) 574 of the camera 570. As shown in FIG. 6, the lens 572 of the camera 570 is oriented such that the marker 560 attached to the first end-effector 213-1 of robotic arm 210-1 falls or is captured within the FoV 574 of camera 570. In this configuration, the visual identifiers 562 of the marker 560 may be captured by the camera 570 in images or image data. The image data captured by the camera 570 may be provided from the camera 570 to the computer system 580 for processing thereby. Additionally, it may be understood that the camera 570 may be supported by a support structure (not shown in FIG. 6) whereby the camera 570 remains stationary with respect to a global coordinate frame (indicated by arrow 590 in FIG. 6). In some instances, the base 554 may also be maintained stationary with respect to the global coordinate frame 590 while the medical tools 212 of robotic arms 210-1 and 210-2 are permitted to move in accordance with one or more DoFs relative to the global coordinate frame 590.

Although only the single marker 560 is shown in FIG. 6, in other embodiments, vibration monitoring system 550 may include two or more markers 560 or any other number of the markers 560 applicable to the purpose of this disclosure. For instance, in an embodiment, the first marker 560 may be coupled to the end-effector 213-1 of robotic arm 210-1 while a second marker 560 is coupled to a HID of the vibration monitoring system 550 (e.g., coupled to a handle 484 of the HID 480 shown in FIG. 4). In this manner, the actual positions of both the first marker 560 coupled to end-effector 213-1 and the second marker 560 coupled to the HID may each be monitored or detected and the resulting position error may be based on both actual positions. In at least some embodiments, movement of the HID (e.g., movement of the handle 484 of HID 480) is intended to mimic or mirror the movement of the end-effector controlled by the respective HID. In other words, the HID 480 may be manipulated (e.g., manually by an operator of the HID) along a given trajectory in order to induce a similar corresponding motion in the end-effector controlled by the HID. Thus, an expected position of the end-effector may be determined from the actual position of the HID (e.g., captured via monitoring of the second marker coupled to the HID) as the HID is moved along an intended trajectory.

The computer system 580 of a vibration monitoring system 550 generally includes a processor 582 and a non-transitory storage medium or memory device 584 connected to the processor 582. The memory device 584 is encoded with instructions executable by the processor 582. Additionally, in some embodiments, the computer system 580 is connected or otherwise in signal communication with a processor (e.g., processor 380) of the surgical robot 200.

In some embodiments, the memory device 584 stores a predefined intended test trajectory of the end-effector 213 of the medical tool 212 to which the marker 560 is coupled, where it may be understood that the intended test trajectory of the end-effector 213 corresponds to an intended test trajectory of the marker 560 given that the position of the marker 560 is coincident with the position of the end-effector 213 of medical tool 212. The intended test trajectory of the end-effector 213 may be predefined such that the intended positions of the end-effector 213, along the intended trajectory, are also known and predefined. For example, the intended positions of the end-effector 213 along the intended trajectory may also be stored in the memory device 584.

It may be understood that the intended test trajectory may be executed automatically by the robotic arm 210-1 using a processor (e.g. processor 380) of the surgical robot 200 whereby the HID of vibration monitoring system 550 is activated by the processor in an attempt to actuate end-effector 213 of the medical tool 212 along the intended test trajectory. For example, a memory device (e.g., memory 382) of the surgical robot 200 may store a kinematic model of surgical robot 200 whereby an intended trajectory of end-effector 213 of the medical tool 212 of the respective robotic arm 210 may be encoded, using the kinematic model, as a series of control inputs to the joints 304 of the respective robotic arm 210 such that the end-effector 213, absent vibration in the robotic arm 210 or collision with an external object, may be expected to travel along the intended trajectory. In some embodiments, the kinematic model of surgical robot 200 comprises a forward kinematic model which utilizes kinematic equations of the surgical robot 200 to determine the position of an end-effector (e.g., end-effector 213) from known parameters of the surgical robot 200.

In some embodiments, the HID of the surgical robot 200 may be commanded automatically by the computer system 580 to activate the robotic arm 210-1 such that the series of control inputs corresponding to the intended test trajectory are applied by the HID to the joints 304 (indicated as 304-1 through 304-6) of the robotic arm 210-1. In other embodiments, the HID of the surgical robot 200 is commanded by a user of the surgical robot 200 to activate the robotic arm 210-1 such that the series of control inputs corresponding to the intended test trajectory are applied to the joints 304 of the robotic arm 210-1.

In certain embodiments, in response to the activation of the robotic arm 210-1 by the processor of the surgical robot 200 to execute the intended test trajectory, the end-effector 213 of robotic arm 210-1 begins to travel along an actual trajectory associated with, but not the same as, the intended test trajectory. As will be discussed further herein, the actual trajectory of end-effector 213 may depart from the intended test trajectory due to, for example, vibration in the surgical robot 200, including self-excitation of the robotic arm 210-1 in response to the activation and resulting motion of the robotic arm 210-1.

In certain embodiments, as the end-effector 213 of the robotic arm 210-1 travels along the actual trajectory of the end-effector 213, the actual position of the end-effector 213 is monitored or tracked by the vibration monitoring system 550. For example, the camera 570 of the vibration monitoring system 550 may monitor or track the actual position of end-effector 213. In this manner, the actual position of the end-effector 213 along its actual trajectory is captured in image data by the camera 570. It may be understood that the camera 570 may be positioned, and the intended test trajectory may be configured, such that marker 560 remains within the FoV 574 of camera 570 over the entire actual trajectory of the end-effector 213/marker 560. Thus, the entire actual trajectory of the end-effector 213/marker 560 may be captured by the camera 570 as image data and supplied to the computer system 580 of vibration monitoring system 550.

As described above, the actual trajectory of the end-effector 213 coupled to the robotic arm 210-1 may depart from the intended test trajectory of the end-effector 213 due to vibration in the surgical robot 200. In some embodiments, the vibration monitoring system 550 may monitor said vibration by comparing the actual trajectory of end-effector 213 with the intended test trajectory of end-effector 213.

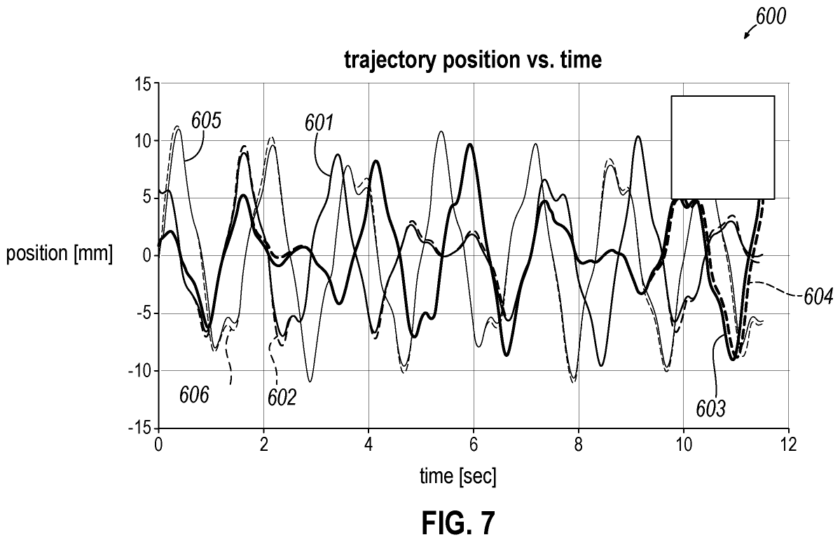

In some embodiments, position data of the marker 560 is extracted by the computer system 580 (e.g., via a computer vision algorithm executed by the processor 582 thereof) from the image data provided to the computer system 580 from the camera 570. Particularly, the actual position of the marker 560 in the X, Y, and Z directions may be captured by the computer system 580 from the image data received from camera 570. For example, referring briefly to FIG. 7, an exemplary graph 600 shows the actual position of marker 560 along the actual trajectory thereof along with the intended position of marker 560 along the intended test trajectory thereof. Particularly, graph 600 illustrates exemplary intended positions 601, 603, and 605 along the X, Y, and Z directions, respectively. Additionally, graph 600 illustrates exemplary actual positions 602, 604, and 606 along the X, Y, and Z directions, respectively. It may be noted from graph 600 in this example that while the actual positions 602, 604, and 606 and intended positions 601, 603, and 605 of marker 560 along the X, Y, and Z directions substantially coincide, the actual positions 602, 604, and 606 of marker 560 vary to a degree (with the magnitude of variance changing over time) from the intended positions 601, 603, and 605 of marker 560 due, in at least in part, to vibration such as self-excitation in the surgical robot 200.

It may be understood that in order to properly match or overlay the actual positions 602, 604, and 606 of the marker 560 with the intended positions 601, 603, and 605, the actual positions 602, 604, and 606 may be time synced (e.g., due to latency inherent in the control of surgical robot 200) by the computer system 580 with the intended positions 601, 603, and 605 to temporally align the actual trajectory of marker 560 with the intended test trajectory of marker 560. In some embodiments, the actual positions 602, 604, and 606 of marker 560 may be time synced to match the intended positions 601, 603, and 605 of marker 560 using a predefined registration trajectory of the marker 560. Additionally, in some embodiments, the actual positions 602, 604, and 606 may be interpolated to temporally match the intended positions 601, 603, and 605. Further, in certain embodiments, the intended positions 601, 603, and 605 are scaled to match the actual positions 602, 604, and 606 in view of the particular scaling settings of the surgical robot 200.

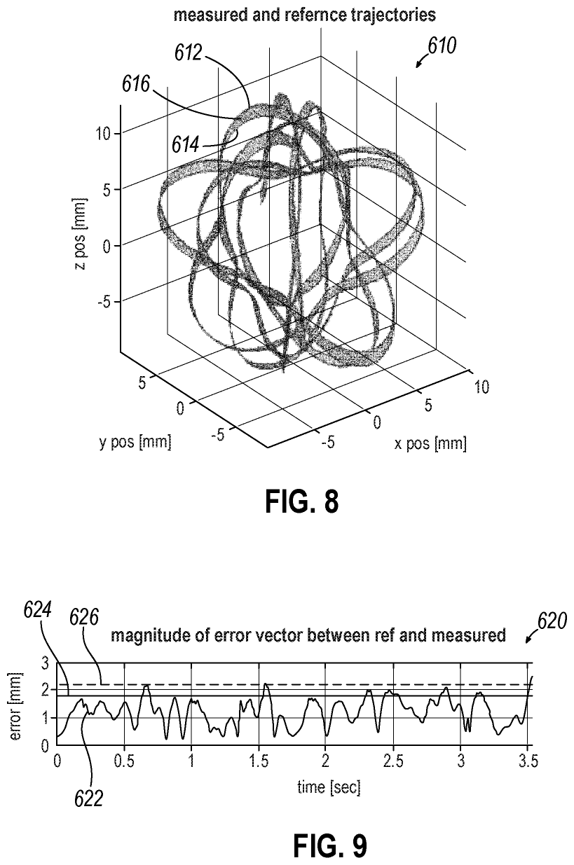

In some embodiments, in addition to time syncing the actual trajectory of marker 560 gleaned from the image data received from camera 570, the computer system 580 may also match the coordinate frames of the actual trajectory of marker 560 with the intended test trajectory of marker 560 whereby the actual trajectory of marker 560 may be overlaid with the intended test trajectory of marker 560 in three-dimensional (3D) space. For example, and referring briefly to FIG. 8, a graph 610 is shown illustrating an exemplary actual trajectory 612 of the marker 560 along with an exemplary intended test trajectory 614 of the marker 560 in 3D space with a common coordinate frame (e.g., global coordinate frame 590 shown in FIG. 6). In this example, the band extending orthogonally between the actual trajectory 612 and intended test trajectory 614 represents, in some embodiments, the position error 616 of marker 560 in the form of a continuously varying position error vector. In other embodiments, the position error of marker 560 corresponding to the difference between an actual position and an expected position thereof may be determined in ways other than comparing an actual trajectory of the marker 560 with an intended test trajectory of the marker 560.

In certain embodiments, the coordinate frames of the actual and intended test trajectories of the marker 560 are matched by point cloud matching a first plurality of points or point cloud in 3D space representing the actual positions of marker 560 with a second plurality of points or point cloud representing the intended test positions of marker 560.

In some embodiments, the actual and intended test trajectories may be broken down into piecewise increments or time segment having a predefined duration such as, for example, 0.45 seconds (corresponding to twice the duration of a typical person's visual reaction time), in some embodiments. These distinct time segment may then be point cloud matched and then added back together to form, for example, the point cloud matched actual trajectory 612 and intended test trajectory 614 of marker 560. In some embodiments, a position error is determined for each given time segment. For instance, a first position error corresponding to the first time segment may be determined by comparing an actual position and an expected position each corresponding to the first time segment. Each time segment may be treated independently such that the initial expected position of the marker 560 for a second time segment immediately following the first time segment corresponds to the actual position of marker 560 at the conclusion of the first time segment, and the position error for the second time segment corresponds to the difference between a final actual position of the marker 560 and a final expected position of the marker 560 at the conclusion of the second time segment.

In some embodiments, following the linking of the coordinate frames of the actual and intended test trajectories of the marker, the position error (e.g., position error 616) obtained therefrom may be compared with a predefined reference threshold or standard to determine the suitability of the surgical robot (e.g., surgical robot 200) for performing operations. Particularly, it may be determined that, in response to the position error exceeding the reference standard, the surgical robot is unsuitable for operation due to an excessively larger position error between the actual and expected positions (e.g., the actual and intended test trajectories) driven by unsatisfactorily excessive vibration in the surgical robot. In some embodiments, surgical robots determined to have an excessively large position error may be repaired, redressed, refurbished, etc., and then retested using vibration monitoring system 550 to again determine the surgical robot's suitability for operation.

As an example, and referring to FIG. 9, a graph 620 is shown illustrating position error magnitude 622 (varying with respect to time) along with at least a 90th percentile position error 626 (comprising a single value) compared to a reference standard 624 (comprising a single value). As an exemplary embodiment, it may be understood that the at least 90th percentile position error 626 may be obtained from the position error magnitude 622 across the respective actual trajectory of the marker (e.g., marker 560). By comparing the at least 90th percentile position error 626 with the reference standard 624 it may be determined whether there is at least 90% likelihood as to whether the position error of the marker falls above or below the reference standard 624. However, while in this example the at least 90th percentile position error 626 is compared with the reference standard 624 to determine the suitability of the respective surgical robot for operation, it may be understood that in other embodiments other positions errors (e.g., mean position error, median position error, 75th percentile position error, 95th percentile position error, 99.7th percentile position error) may instead be compared with the reference standard 624 to determine the suitability of the surgical robot for operation.

Referring again to FIG. 6, as described above, vibration monitoring system 550 may, in some embodiments, be used to determine the position error in marker 560 by actuating end-effector 213 (e.g., via driving the robotic arm 210-1) coupled therewith and comparing an expected position of the marker 560 (e.g., based on a kinematic model of the surgical robot 200 and an input provided by a HID controlling the end-effector 213) with an actual position of marker 560. In some embodiments, end-effector 213 may be actuated along an intended test trajectory where an actual trajectory of the marker 560 may be compared with the intended test trajectory. In this manner, the magnitude of the resulting position error between the actual trajectory and the intended trajectory may result at least partially from self-excitation of the robotic arm 210-1 in response to being driven along the intended test trajectory.

In addition to determining position error resulting from self-excitation in the robotic arm 210-1 in an activated state as described above, vibration monitoring system 550 may also be utilized to determine position error in the stationary robotic arm 210-1 (or robotic arm 210-2) from crosstalk produced from a moving robotic arm 210-2. Particularly, as with the technique for monitoring self-excitation described above, initially the marker 560 may be coupled to end-effector 213-1. However, instead of actuating end-effector 213-1 (e.g., via driving the robotic arm 210-1 along an intended test trajectory), the robotic arm 210-1 remains stationary in the stationary state while end-effector 213-2 coupled to the robotic arm 210-2 (which does not include the marker 560 in this example) is actuated such as via an input provided by an HID associated with robotic arm 210-2. The end-effector 213-2 may be actuated in ways other than providing an input by an HID. For example, in some embodiments, the surgical table (e.g., patient platform 202 shown in FIG. 2) upon which end-effector 213-1 is supported may be moved to thereby actuate end-effector 213-2. In other embodiments, the the end-effector 213-2 may be moved manually by an operator of the surgical robot 200. It may also be understood that the intended test trajectory for monitoring crosstalk may vary in configuration from the intended test trajectory for monitoring self-excitation. Additionally, the intended test trajectory for monitoring crosstalk may not be predefined (e.g., it may be spontaneously inputted to the robotic arm 210-2 by a user of vibration monitoring system 550).

In some embodiments, with the robotic arm 210-2 in the activated state driven along the intended test trajectory, the position of the marker 560 is monitored by camera 570 and captured thereby in image data in a manner similar to the technique for monitoring self-excitation described above. In other embodiments, sensors other than cameras (e.g., camera 570) may be utilized for tracking the position of the end-effector 213 such as, for example, accelerometers, encoders, electromagnetic tracking, and the like. In this exemplary embodiment, the resulting image data captured by camera 570 is received by the computer system 580 and the position of the marker 560 may be extracted by the computer system 580 (e.g., via a computer vision algorithm executed by the processor 582 thereof) from the received image data.

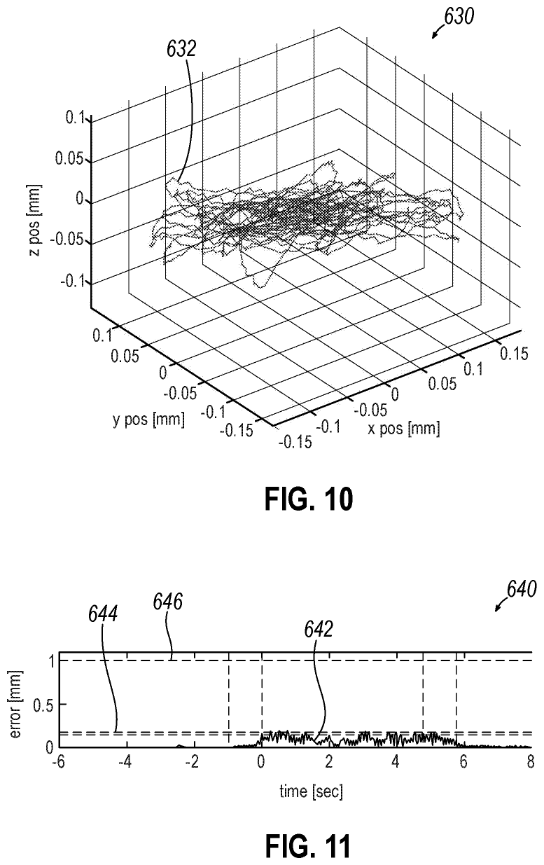

For example, and referring to FIG. 10, a graph 630 is shown illustrating an exemplary actual trajectory 632 of the marker 560 in 3D space (e.g., with reference to global coordinate frame 590) as the robotic arm 210-2 is conducted along the intended test trajectory. It may be noted from graph 630 that the actual trajectory 632 of marker 560 does not remain stationary with reference to global coordinate frame 590 as the robotic arm 210-2 is conducted along the intended test trajectory, and instead, the actual trajectory 632 of marker 560 travels erratically in 3D space.

Referring to FIG. 11, a graph 640 is shown illustrating an exemplary position error magnitude 642 obtained from the actual trajectory 632 shown in FIG. 10. For example, the position error magnitude 642 may be obtained from the actual trajectory 632 by comparing the actual trajectory 632 with an intended trajectory (corresponding in this example to a fixed, stationary position of the marker 560) of the marker 560, such as by determining the magnitude of each vector obtained between the plurality of actual positions of the marker 560 forming actual trajectory 632 (which may form or define a point cloud) and the intended stationary position of the marker 560. In addition to position error magnitude 642, graph 640 illustrates at least a 90th percentile position error 644 (comprising a single value) and an exemplary reference threshold or standard 646 (comprising a single value), the at least 90th percentile position error 644 being obtained from the position error magnitude 642.

As with the technique for monitoring self-excitation described above, the fitness of a surgical robot for operation may be determined by comparing the at least 90th percentile position error 644 with the reference standard 646. Particularly, a surgical robot having at least a 90th percentile position error 644 exceeding the reference standard 646 may be deemed unsuitable for operation. In some embodiments, surgical robots determined to have an excessively large position error may be repaired, redressed, refurbished, reconfigured, recalibrated, etc., and then retested using vibration monitoring system 550 to again determine the surgical robot's suitability for operation. Additionally, it may be understood that vibration monitoring system 550 may be used to monitor both self-excitation and crosstalk in a surgical robot (e.g., surgical robot 200 shown in FIG. 6) as separate tests as part of a battery of tests for determining the fitness of the respective surgical robot for operation where failure of any of the given battery of tests may deem the surgical robot unsuitable for operation.

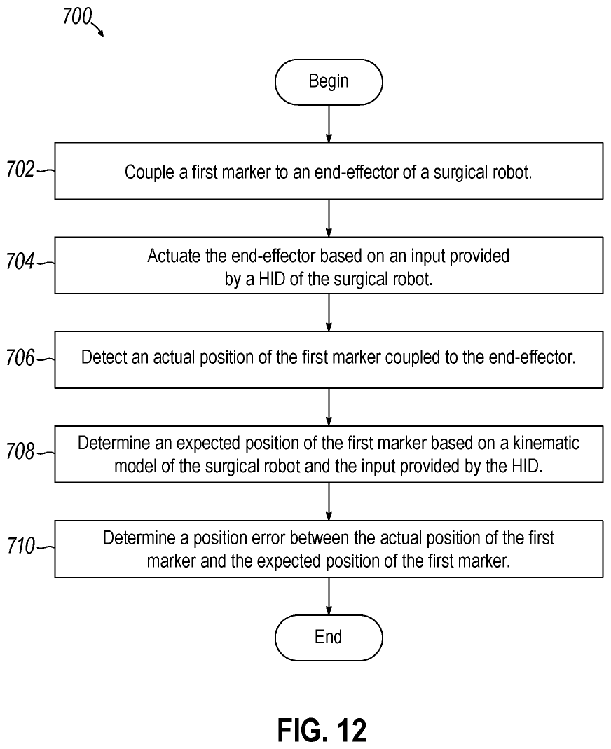

Referring to FIG. 12, a flowchart for a method 700 at least partially performed by one or more processors (e.g., processor 582) of a system (e.g., vibration monitoring system 550) for monitoring vibration in a surgical robot. In some embodiments, the vibration monitoring system may be part of, or incorporate features of, a surgical robot (e.g., surgical robot 200). Additionally, the vibration monitoring system includes memory (e.g., memory device 584) storing instructions for execution by the one or more processors.

Initially at step 702, method 700 includes coupling a first marker to an end-effector of a surgical robot. In some embodiments, step 702 comprises coupling the marker 560 (shown in FIG. 6) to end-effector 213 of the medical tool 212. For example, the marker may be manually coupled to the end-effector by a user. Alternatively, the marker may be coupled to the robotic arm at locations other than the end-effector thereof, such as to a link or joint of the robotic arm.

At step 704, method 700 includes actuating the end-effector based on an input provided by a HID of the surgical robot (e.g., HID 480 shown in FIG. 4). In some embodiments, step 704 comprises actuating (e.g., moving, transporting, displacing) the end-effector 213 of the medical tool 212 shown in FIG. 6.

At step 706, method 700 includes detecting an actual position of the first marker coupled to the end-effector. In certain embodiments, step 706 comprises detecting an actual position of the marker 560 shown in FIG. 6 as end-effector 213 of the medical tool 212 as the end-effector 213 is actuated (e.g., moved by the robotic arm 210-1 shown in FIG. 6)

At step 708, method 700 includes determining an expected position of the first marker based on a kinematic model of the surgical robot and the input provided by the HID (e.g., HID 480). In some embodiments, step 708 comprises determining an expected position of the marker 560 (shown in FIG. 6) based on a kinematic model (e.g., a forward kinematic model) of the surgical robot 200 (shown in FIG. 6) and the input provided by the HID. At step 710, method 700 includes determining a position error between the actual position of the first marker and the expected position of the first marker. In some embodiments, step 710 comprises determining a position error between the actual position of the marker 560 (shown in FIG. 6) and the expected position of the marker 560.

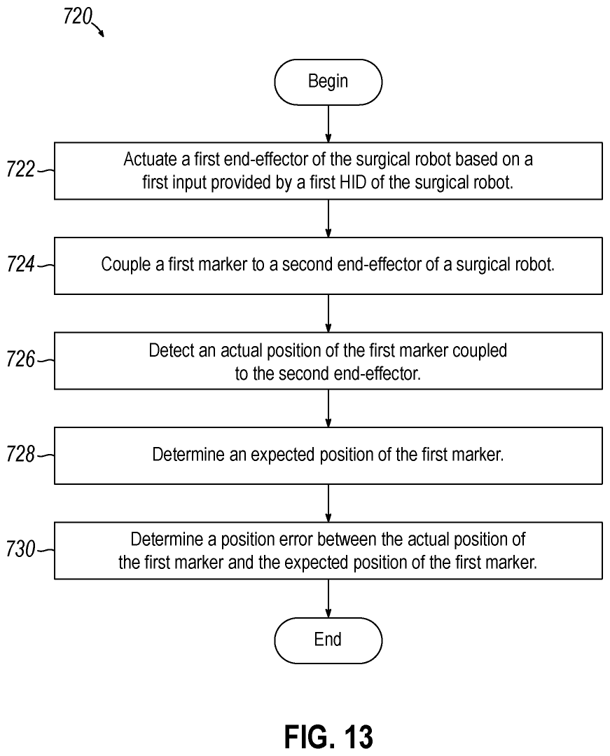

Referring to FIG. 13, a flowchart for another method 720 at least partially performed by one or more processors (e.g., processor 582) of a system (e.g., vibration monitoring system 550) for monitoring vibration in a surgical robot. In some embodiments, the vibration monitoring system may be part of, or incorporate features of, a surgical robot (e.g., surgical robot 200). Additionally, the vibration monitoring system includes memory (e.g., memory device 584) storing instructions for execution by the one or more processors.

Initially at step 722, method 720 includes actuating a first end-effector of a surgical robot based on a first input provided by a first HID (e.g., HID 480 shown in FIG. 4) of the surgical robot. In some embodiments, step 722 comprises actuating (e.g., moving, transporting, displacing) the end-effector 213-2 coupled to robotic arm 210-2 of surgical robot 200 (shown in FIG. 6) based on a first input provided by a first HID of surgical robot 200.

At step 724, method 720 includes coupling a first marker to a second end-effector of the surgical robot. In some embodiments, step 724 comprises coupling the marker 560 (shown in FIG. 6) to end-effector 213-1 coupled to robotic arm 210-1 (shown in FIG. 6). For example, the marker may be manually coupled to the end-effector by a user of the surgical robot. Alternatively, the marker may be coupled to the robotic arm at locations other than the end-effector thereof, such as to a link or joint of the robotic arm.

At step 726, method 720 includes detecting an actual position of the first marker coupled to the second end-effector. In certain embodiments, step 726 comprises monitoring a position of the marker 560 shown in FIG. 6 as end-effector 213-2 coupled to robotic arm 210-2 is actuated. For instance, end-effector 213-2 may be actuated by actuating at least one of the robotic arm 210-2 and the base 554 (shown in FIG. 6) such as by moving or displacing the base 554.

At step 728, method 720 includes determining an expected position of the first marker. In some embodiments, step 728 comprises determining an expected position of the marker 560 coupled to robotic arm 210-1 (shown in FIG. 6). In certain embodiments, step 728 comprises determining an expected position of the marker 560 based on a kinematic model (e.g., a forward kinematic model) of the surgical robot 200 (shown in FIG. 6) and an input provided by the first HID (e.g., HID 480 shown in FIG. 4) of the surgical robot 200.

At step 730, method 720 includes determining a position error between the actual position of the first marker and the expected position of the first marker. In some embodiments, step 730 comprises determining a position error between the actual position of marker 560 (shown in FIG. 6) and the expected position of marker 560.

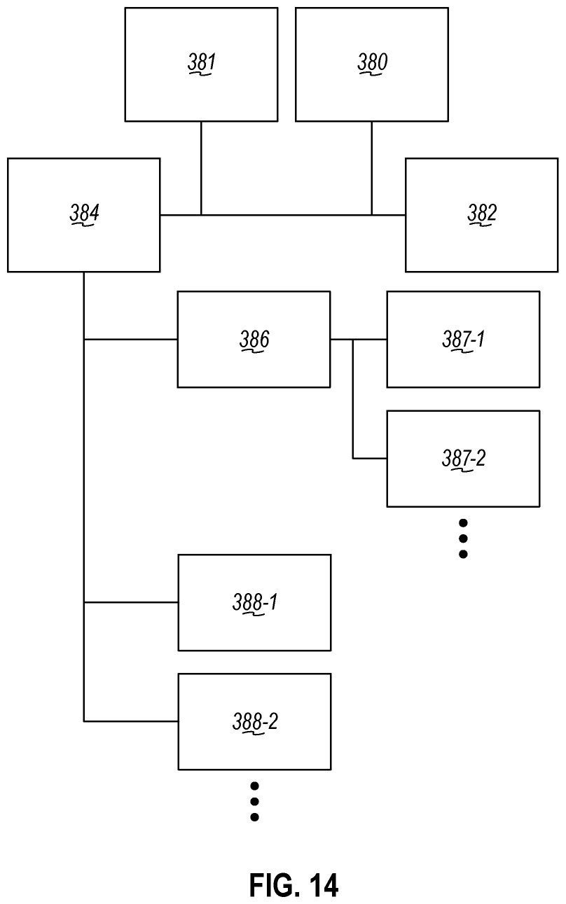

Referring to FIG. 14, a schematic diagram illustrating electronic components of a surgical robot is shown in accordance with some embodiments.

The robotic medical system includes one or more processors 380, which are in communication with a computer-readable storage medium 382 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 2, 4, 5A-5C, 6). The one or more processors 380 are also in communication with an input/output controller 384 (via a system bus or any suitable electrical circuit). The input/output controller 384 receives sensor data from one or more sensors 388-1, 388-2, etc., and relays the sensor data to the one or more processors 380. The input/output controller 384 also receives instructions and/or data from the one or more processors 380 and relays the instructions and/or data to one or more actuators, such as first motors 387-1 and 387-2, etc. In some embodiments, the input/output controller 384 is coupled to one or more actuator controllers 386 and provides instructions and/or data to at least a subset of the one or more actuator controllers 386, which, in turn, provide control signals to selected actuators. In some embodiments, the one or more actuator controllers 386 are integrated with the input/output controller 384 and the input/output controller 384 provides control signals directly to the one or more actuators 387-1, 387-2, etc. (without a separate actuator controller). Although FIG. 14 shows that there is one actuator controller 386 (e.g., one actuator controller for the entire medical robotic system, in some embodiments, additional actuator controllers may be used (e.g., one actuator controller for each actuator, etc.). In some embodiments, the one or more processors 380 are in communication with one or more displays 381 for displaying information as described herein.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example Combination 1: A method, may include: coupling a first marker to an end-effector of a surgical robot; actuating the end-effector based on an input provided by a human interface device (HID) of the surgical robot; detecting an actual position of the first marker coupled to the end-effector; determining an expected position of the first marker based on a kinematic model of the surgical robot and the input provided by the HID; and determining a position error between the actual position of the first marker and the expected position of the first marker.

Example Combination 2: The method of Example Combination 1, further may include: terminating the actuating of the end-effector in response to the position error exceeding a predefined threshold.

Example Combination 3: The method of Example Combination 1 or Example Combination 2, further may include: coupling a second marker to the HID.

Example Combination 4: The method of any one of Example Combinations 1-3, where the input from the HID is generated in response to moving the HID along a predefined trajectory.

Example Combination 5: The method of any one of Example Combinations 1-4, where the first marker may include an optical marker, and the detecting of the actual position may include optically monitoring by an optical device the position of the optical marker as the end-effector is actuated.

Example Combination 6: The method of any one of Example Combinations 1-5, where the end-effector moves along a predefined test trajectory based on the input provided by the HID.

Example Combination 7: The method of any one of Example Combinations 1-6, where the expected position corresponds to a position along a predefined intended trajectory of the first marker.

Example Combination 8: The method of any one of Example Combinations 1-7, further may include: determining at least a 90th percentile of the position error across an actual trajectory of the first marker may include the actual position.

Example Combination 9: The method of any one of Example Combinations 1-8, further may include: defining an error metric corresponding to (Eₘₑₐₙ+2xE_{SD}), where Eₘₑₐₙ may include a mean of the position error, x may include a predefined constant, and E_{SD} may include a standard deviation of the position error; and determining an acceptability of a vibration induced in the surgical robot by determining whether the error metric meets or exceeds a predefined error threshold

Example Combination 10: The method of any one of Example Combinations 1-9, further may include: determining an acceptability of a vibration induced in the surgical robot by determining whether the position error meets or exceeds a predefined error threshold.

Example Combination 11: The method of any one of Example Combinations 1-10, further may include: inferring a position error of the end-effector based on the determined position error between the actual position of the first marker and the expected position of the first marker.

Example Combination 12: The method of any one of Example Combinations 1-11, where: the actual position may include a first actual position of the first marker corresponding to a first time segment occurring as the end-effector is actuated; and the expected position may include a first expected position corresponding to the first time segment and based on the kinematic model, and the input provided by the HID during the first time segment.

Example Combination 13: The method of any one of Example Combinations 1-12, further may include: determining a second expected position of the first marker corresponding to a second time segment and based on the kinematic model and and the input provided by the HID during the second time segment, where the second time segment follows the first time segment; and determining a second position error between a second actual position of the first marker corresponding to the second time segment and the second expected position of the first marker.

Example Combination 14: A system for monitoring vibration induced in a surgical robot, the system may include an end-effector having a first marker coupled thereto; a human interface device (HID) for actuating the end-effector; and a processor communicatively coupled to the HID, where the processor is configured to: actuate the end-effector based on an input provided by the HID; detect an actual position of the first marker attached to the end-effector; determine an expected position of the first marker based on a kinematic model of the surgical robot and the input provided by the HID; and determine a position error between the actual position of the first marker and the expected position of the first marker.

Example Combination 15: The system of Example Combination 14, where the processor is configured to: determine at least a 90th percentile of the position error across an actual trajectory of the first marker may include the actual position.

Example Combination 16: The system of Example Combination 14 or Example Combination 15, where the processor is configured to: define an error metric corresponding to (Eₘₑₐₙ+KE_{SD}), where Eₘₑₐₙ may include a mean of the position error, K may include a predefined constant, and E_{SD} may include a standard deviation of the position error; and determine an acceptability of a vibration induced in the surgical robot by determining whether the error metric meets or exceeds a predefined error threshold.

Example Combination 17: The system of any one of Example Combinations 14-16, where the processor is configured to: determine an acceptability of the vibration induced in the surgical robot by determining whether the position error meets or exceeds a predefined error threshold.

Example Combination 18: The system of any one of Example Combinations 14-17, where the first marker may include an optical marker.

Example Combination 19: The system of any one of Example Combinations 14-18, further may include a second marker coupled to the HID.

Example Combination 20: The system of any one of Example Combinations 14-19, where: the first marker may include an optical marker; and where the processor is configured to: monitor by an optical device communicatively coupled to the processor the actual position of the optical marker as the end-effector is actuated.

Example Combination 21: A method, may include: actuating a first end-effector of a surgical robot based on a first input provided by a first human interface device (HID) of the surgical robot; coupling a first marker to a second end-effector of a surgical robot; detecting an actual position of the first marker coupled to the second end-effector; determining an expected position of the first marker; and determining a position error between the actual position of the first marker and the expected position of the first marker.

Example Combination 22: The method of Example Combination 21, where the actuating of the first end-effector may include at least one of moving a surgical table to which the first end-effector is coupled and repositioning a robotic arm to which the first end-effector is coupled.

Example Combination 23: The method of Example Combination 21 or Example Combination 22, where the expected position may include a single stationary position of the first marker.

Example Combination 24: The method of any one of Example Combinations 21-23, further may include: actuating the second end-effector based on a second input provided by a second HID of the surgical robot, where the expected position is based on a kinematic model of the surgical robot and the second input provided by the second HID.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions for determining whether a tool is within or outside a surgical field of view provided by a camera or scope and rendering one or more indicators representing positions or directions of one or more medical tools described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or implementations.

As used herein, the term “and/or” encompasses any combination of listed elements. For example, “A, B, and/or C” includes the following sets of elements: A only, B only, C only, A and B without C, A and C without B, B and C without A, and a combination of all three elements, A, B, and C.

The various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the scope of the disclosure. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present disclosure is not intended to be limited to the embodiments shown herein but is to be accorded the widest scope consistent with the principles and novel features disclosed herein.

What is claimed is:

1. A method, comprising:
actuating an end-effector of a surgical robot based on an input provided by a human interface device (HID) of the surgical robot;
detecting an actual position of a first marker coupled to the end-effector;
determining an expected position of the first marker based on a kinematic model of the surgical robot and the input provided by the HID; and
determining a position error between the actual position of the first marker and the expected position of the first marker.

2. The method of claim 1, further comprising:
terminating the actuating of the end-effector in response to the position error exceeding a predefined threshold.

3. The method of claim 1, further comprising:
coupling a second marker to the HID.

4. The method of claim 1, wherein the input from the HID is generated in response to moving the HID along a predefined trajectory.

5. The method of claim 1, further comprising coupling the first marker to the end-effector, wherein the first marker comprises an optical marker, and the detecting of the actual position comprises optically monitoring by an optical device the position of the optical marker as the end-effector is actuated.

6. The method of claim 1, wherein the end-effector moves along a predefined test trajectory based on the input provided by the HID.

7. The method of claim 1, wherein the expected position corresponds to a position along a predefined intended trajectory of the first marker.

8. The method of claim 1, further comprising:
determining at least a 90^{th} percentile of the position error across an actual trajectory of the first marker comprising the actual position.

9. The method of claim 1, further comprising:
defining an error metric corresponding to (Eₘₑₐₙ+2xE_{SD}), where Eₘₑₐₙ comprises a mean of the position error, x comprises a predefined constant, and E_{SD} comprises a standard deviation of the position error; and
determining an acceptability of a vibration induced in the surgical robot by determining whether the error metric meets or exceeds a predefined error threshold.

10. The method of claim 1, further comprising:
determining an acceptability of a vibration induced in the surgical robot by determining whether the position error meets or exceeds a predefined error threshold.

11. The method of claim 1, further comprising:
inferring a position error of the end-effector based on the determined position error between the actual position of the first marker and the expected position of the first marker.

12. The method of claim 1, wherein:
the actual position comprises a first actual position of the first marker corresponding to a first time segment occurring as the end-effector is actuated; and
the expected position comprises a first expected position corresponding to the first time segment and based on the kinematic model, and the input provided by the HID during the first time segment.

13. The method of claim 12, further comprising:
determining a second expected position of the first marker corresponding to a second time segment and based on the kinematic model and and the input provided by the HID during the second time segment, wherein the second time segment follows the first time segment; and
determining a second position error between a second actual position of the first marker corresponding to the second time segment and the second expected position of the first marker.

14. A system for monitoring vibration induced in a surgical robot, the system comprising
an end-effector having a first marker coupled thereto;
a human interface device (HID) for actuating the end-effector; and
a processor communicatively coupled to the HID, wherein the processor is configured to:
actuate the end-effector based on an input provided by the HID;
detect an actual position of the first marker attached to the end-effector;
determine an expected position of the first marker based on a kinematic model of the surgical robot and the input provided by the HID; and
determine a position error between the actual position of the first marker and the expected position of the first marker.

15. The system of claim 14, wherein the processor is configured to:
determine an acceptability of the vibration induced in the surgical robot by determining whether the position error meets or exceeds a predefined error threshold.

16. The system of claim 14, wherein:
the first marker comprises an optical marker; and
wherein the processor is configured to:
monitor by an optical device communicatively coupled to the processor the actual position of the optical marker as the end-effector is actuated.

17. A method, comprising:
actuating a first end-effector of a surgical robot based on a first input provided by a first human interface device (HID) of the surgical robot;
coupling a first marker to a second end-effector of a surgical robot;
detecting an actual position of the first marker coupled to the second end-effector;
determining an expected position of the first marker; and
determining a position error between the actual position of the first marker and the expected position of the first marker.

18. The method of claim 17, wherein the actuating of the first end-effector comprises at least one of moving a surgical table to which the first end-effector is coupled and repositioning a robotic arm to which the first end-effector is coupled.

19. The method of claim 17, wherein the expected position comprises a single stationary position of the first marker.

20. The method of claim 17, further comprising:
actuating the second end-effector based on a second input provided by a second HID of the surgical robot, wherein the expected position is based on a kinematic model of the surgical robot and the second input provided by the second HID.
